(19) United States
(12) Patent Application Publication
(10) Pub. No.: US 2010/0249670 A1
(43) Pub. Date: Sep. 30, 2010

(54) HIGH-POWER MULTIPLE-HARMONIC ULTRASOUND TRANSDUCER

(75) Inventors: Tonee SMITH, Redwood City, CA (US); Justin May, Redwood City, CA (US)

Correspondence Address: Cutera c/o MoFo, 425 Market Street, San Francisco, CA, 94105-2482, US

(73) Assignee: CUTERA, INC., Brisbane, CA (US)

(21) Appl. No.: 12/727,153

(22) Filed: Mar. 18, 2010

Related U.S. Application Data
(60) Provisional application No. 61/161,985, filed on Mar. 20, 2009.

(51) Int. Cl. A61N 7/00

(52) U.S. Cl. 601/2

(57) ABSTRACT

A dermatological treatment device that includes an ultrasound transducer and at least one matching layer mechanically interfaced to an output surface of the transducer. The ultrasonic transducer has a fundamental resonant frequency and the at least one matching layer has a thickness about equal to an odd multiple of a quarter-wavelength of the fundamental frequency. A drive circuit applies an electrical power input to the ultrasonic transducer. The electrical power input has a drive frequency that is an odd harmonic of the fundamental resonant frequency of the ultrasonic transducer, and a drive voltage greater than 55 volts. The electrical power input results in an ultrasonic transducer output with a power intensity greater than 150 W/cm².

### CROSS-REFERENCE TO RELATED APPLICATION

This application claims priority to U.S. Provisional Application Ser. No. 61/161,985 filed Mar. 20, 2009, which is incorporated herein by reference in its entirety for all purposes.

### FIELD OF THE INVENTION

The subject invention generally relates to an ultrasound transducer for treating tissue. More specifically, the invention relates to a high-power multiple-harmonic ultrasonic transducer for wide-area irradiation to inhibit hair growth.

### BACKGROUND

Ultrasound systems have a wide range of applications including, for example, medical procedures for imaging, diagnosis, or treatment of a human body. Using an ultrasonic transducer, energy can be transmitted to adjacent tissue so that the energy can be absorbed by parts of the body.

In some applications, ultrasonic energy can be used to remove unwanted hair from skin tissue. The underlying principal is to use ultrasound radiation to selectively induce damage to the hair structure and thereby retard its ability to regenerate. Typically, the bulb or bulge of the hair follicle is targeted since these features are thought to be involved in the regenerative process of hair growth. These features are commonly located several millimeters below the skin surface.

To produce the irradiation exposure necessary to damage a hair follicle, relatively high powers are required. Previous methods achieved high-power exposure by focusing an ultrasonic beam to a point of high intensity on a hair follicle below the surface of the skin. Since the hair follicle is typically several millimeters below the skin surface, the practical limits of beam focusing require the beam radius on the skin surface to be less than several millimeters wide. One drawback to such a system is that only small areas of the skin can be treated at the same time.

The small treatment area of ultrasound focused-beam techniques puts it at a comparative disadvantage to existing light-based and large-area treatment methods. For example, existing light-based technologies are capable of treating large areas in a short period of time. Common areas for light-based hair removal treatments include the axilla (armpit), arms, legs, back, chin, and pubic areas where the hair density ranges from 50 to 500 follicles/cm². (Helen R. Bickmore, Milady's Hair Removal Techniques: A Comprehensive Manual, Thompson Learning Inc. (2004).) Using light-based technologies, the typical treatment area may range from 1 to more than 100 cm², and the treatments can be performed at speeds up to 3 cm²/sec. As a result, using light-based technologies, 50 to 50,000 hairs may be treated in a period between 1 and 33 seconds.

To compete with existing light-based hair removal techniques, it is more desirable to use a broad-area ultrasound transducer to irradiate a larger portion of the skin. One such system is disclosed in U.S. Publication 2008/0183110 assigned to the same assignee herein and incorporated by reference.

Because a broad-area system treats a larger area than focused devices, there is a need to drive the transducers to a higher power. One solution to producing a relatively high-power ultrasonic beam is to drive a transducer using a high voltage. The maximum voltage is dependent on, in part, the dielectric strength of the piezoelectric material and the thickness of the piezoelectric element used in the transducer. Exceeding the maximum voltage may cause the piezoelectric element to break down, resulting in a failure of the transducer. In general, if a thicker piezoelectric element is used, more power can be delivered to the skin.

However, the fundamental resonant frequency of the piezoelectric element is inversely proportional to its thickness. That is, the fundamental operating frequency for a thicker element is lower than that of a thinner element. Accordingly, if high powers are desired, thicker elements operating at lower frequencies must be used. It is desirable to develop an ultrasonic system providing higher operating frequencies at high power without destroying the transducer.

### SUMMARY

In one aspect of the present invention a hand-held dermatological treatment device comprises: an ultrasonic transducer, at least one matching layer mechanically interfaced to an output surface of the ultrasonic transducer, and a drive circuit for applying an electrical power input to the ultrasonic transducer. The ultrasound transducer has a fundamental resonant frequency and the at least one matching layer has a thickness about equal to an odd multiple of a quarter wavelength of the fundamental frequency. The electrical power input provided by the drive circuit has a drive frequency that is an odd harmonic of the fundamental resonant frequency of the ultrasonic transducer, and a drive voltage greater than 55 volts. The electrical power input results in an ultrasonic transducer output with a power intensity greater than 150 W/cm².

In some aspects, the drive circuit is configured to selectively apply at least two odd harmonic frequencies of the fundamental resonant frequency of the ultrasonic transducer.

In some aspects, the electrical power input is applied for less than 100 milliseconds.

In some aspects, the odd harmonic of the fundamental frequency is between 5 and 20 MHz.

In some aspects, the fundamental frequency is 5 MHz or less and the odd harmonic of the fundamental frequency is 5 MHz or higher.

In some aspects, the odd harmonic frequency is between 5 and 20 MHz and the electrical power input is applied for less than 100 milliseconds.

One aspect of the present invention includes a method of removing hair from a person's skin with ultrasound energy, comprising the steps of: positioning a dermatological treatment device against the skin, said device including an ultrasound transducer, said transducer having a fundamental resonant frequency; and driving the transducer with an electrical input having a frequency that is an odd harmonic frequency of the fundamental frequency.

### DESCRIPTION OF THE FIGURES

FIG. 1 depicts an embodiment of portions of a high-power, broad-area ultrasonic tissue treatment device.

FIG. 2 depicts a high-voltage failure of a piezoelectric transducer.

FIG. 3 depicts a full-range impedance scan of a piezoelectric element with a 2.25 MHz fundamental resonant frequency.

FIG. 4 depicts a third harmonic resonance peak.

FIG. 5 depicts a fifth harmonic resonance peak.

FIG. 6 depicts an exemplary ultrasonic transducer system.

The figures depict one embodiment of the present invention for purposes of illustration only. One skilled in the art will readily recognize from the following discussion that alternative embodiments of the structures and methods illustrated herein can be employed without departing from the principles of the invention described herein.

### DETAILED DESCRIPTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

An ultrasonic transducer may be used to treat a portion of tissue using ultrasonic energy irradiation. In the field of dermatology, skin tissue may be treated using energy produced by an ultrasonic transducer that transmits an acoustic-energy beam through the surface of the skin. An ultrasonic transducer is useful for performing a variety of treatments including, for example, skin laxity, skin wrinkles, and skin hair removal. In a preferred embodiment, an ultrasonic transducer is used to treat unwanted skin hair by damaging the hair follicle by ultrasound-induced thermal or mechanical effects.

In general, an ultrasonic device may be characterized as a device capable of producing displacements at a frequency higher than the audible range of a human ear (frequencies>20,000). Ultrasonic devices typically include a transducer that converts electrical energy into acoustical energy via vibrational motion at ultrasonic frequencies. The ultrasonic vibration is induced by exciting one or more piezoelectric elements of the transducer using an electrical signal. In a preferred embodiment, a high-frequency electrical signal is transmitted to a pair of electrodes coupled to one or more piezoelectric elements, whereby an electric field is established across the one or more piezoelectric elements. The electric field generates a mechanical standing wave at a frequency approximately equal to the frequency of the electrical signal. The displacement or amplitude of the mechanical standing wave is determined, in part, by the voltage difference between the pair of electrodes. In a preferred embodiment, the piezoelectric element is mechanically coupled to a transmitter mass having an applicator surface designed to transmit the acoustic energy to a portion of the body. Via the transmitter mass, the mechanical standing wave is able to transmit acoustic energy through a medium (e.g., skin tissue) to a target region.

### 1. Broad-Area Ultrasonic Transducer Device

To facilitate treatment, an ultrasonic transducer may have a transmitter mass having an applicator surface that is placed in contact with a portion of the body. In one embodiment, the transducer produces a broad, unfocused (or weakly focused) beam onto an area of skin tissue such that many hairs are within the beam cross-section and may be irradiated. The area of skin in contact with the applicator surface may also be referred to as the treatment area.

One goal of a preferred embodiment is to optimize the amount of energy absorbed by a hair follicle without damaging surrounding tissue. The energy absorbed in the hair follicle induces a temperature rise in the bulb or bulge of the follicle, which is believed to provide an effective treatment for hair removal. Mechanical effects of the ultrasonic energy may also contribute to an effective treatment. Depending on the frequency and duration of the treatment, the energy intensity required at the hair follicle to achieve hair removal may be between 150 and 1000 W/cm². Because the beam is essentially unfocused, this energy is also the output intensity required at the active element of the transducer (e.g., a piezoelectric disk element). In comparison, the highest-power, focused-beam ultrasound transducers typically produce intensities lower than 4 W/cm² at the active element. For certain medical therapeutic applications, arrays of focused piezoelectric elements are used to produce peak intensities ranging from 300 W/cm² to more than 1 kW/cm² over a relatively broad area (e.g., 1-20 cm²). Other medical applications may use an unfocused beam, but are characterized by relatively low-power intensity. Table 1, below, depicts a survey of beam characteristics for various therapeutic devices.

TABLE 1

| | Frequency Range | Area | Duty | Power Intensity |
|---|---|---|---|---|
| Equipment Type | (MHz) | (cm²) | Factor | (W/cm²) |
| Physiotherapy, | 0.75 to 3.0 | 3 | 1 | Less than 5.0 |
| Continuous Wave | | | | |
| Physiotherapy, | 0.75 to 3.0 | 3 | 0.2 | Less than 1.0 |
| Pulsed | | | | |
| Surgery | 0.5 to 3.0 | 50 | 1 | Less than 4.0 |
| Targeted Drug | 1 to 20 | — | — | Less than 30.0 |
| Delivery | | | | |

The simplicity and increased treatment area of broad-area transducers offer a distinct advantage over focused-beam techniques. The output of a high-power broad-area transducer may be tailored to produce a beam with characteristics that are appropriate for the desired treatment. First, the beam must have sufficient power intensity over a given treatment area to effectuate the tissue treatment. With respect to hair removal, the energy density must have sufficient intensity to cause damage to a significant portion of the hair follicles within the treatment area. Second, the transducer must produce a beam that is able to penetrate the tissue at a sufficient depth to affect the targeted region. For purposes of hair removal, the beam must penetrate several millimeters into the skin tissue to reach the depth of the hair follicles. In some cases, the average depth of the hair follicles is approximately 7 mm.

In some cases, it may be desirable to select both the power intensity and operating frequency in order to tailor the irradiation of a targeted region within the treatment area. However, both the power intensity and the fundamental resonant frequency depend, in part, on the thickness of piezoelectric element used in the transducer. In general, thicker piezoelectric elements are able to produce higher power, but have a lower fundamental resonant frequency. Thus, if the thickness of the piezoelectric element is large enough to produce a beam with sufficient power density, the fundamental resonant frequency may be too low to penetrate the tissue to the depth of the targeted region (e.g., the average depth of the hair follicles). Therefore, it is desirable to provide a system that provides both sufficient power and an appropriate operating frequency for a desired tissue treatment.

### 2. Piezoelectric Element Thickness

A conventional single-element thickness-mode piezoelectric ultrasound transducer has a fundamental resonant frequency (fᵣₑₛ) determined in part by the thickness of the piezoelectric element:

fᵣₑₛ=Nₜ/t_{pzt}, (Equation 1)

where Nₜ is the frequency constant of the dielectric material, t_{pzt} is the thickness of the active portion of the piezoelectric element. The frequency constant (Nₜ) for lead zirconate titanate piezoelectric ceramics ranges between 1750 and 2300 Hz·m. Using the same relationship, the thickness of the piezoelectric element may be determined by selecting a dielectric material frequency constant (Nₜ) and an operating frequency (f):

t_{pzt}=Nₜ/f. (Equation 2)

For example, a 10 MHz PZT-8 element with an Nₜ of 2000 Hz·m would be 200 microns thick.

The thickness of the piezoelectric element limits the maximum drive voltage that can be applied. Depending on the material used, the dielectric strength of the piezoelectric material will vary. One reference places the functional limit of the drive voltage at which device failure occurs at 0.275-0.390 kV/mm. (See, e.g., Browder, L. P. “High-Voltage Lifetime Function of the PZT Ceramic/Gas Insulator Interface in Underwater Sound Transducers,” Naval Research Laboratory Orlando Fla. Underwater Sound Reference Detachment (1980).) For the 10 MHz example, the drive voltage should be limited to 55-78 V to avoid a catastrophic material failure.

However, it may be necessary to use higher-drive voltages for high-power irradiation applications. Given that the piezoelectric efficiency may decrease at higher powers, an input voltage exceeding 230 V peak-to-peak may be required to achieve the acoustic output necessary for a broad-area skin treatment. Furthermore, piezoelectric elements have a tendency to depolarize in negative voltage fields (i.e., an electric field oriented opposite to the piezoelectric poling direction). Thus, for high-power operation, the drive signal may need to be positively biased, as much as doubling the peak positive voltage.

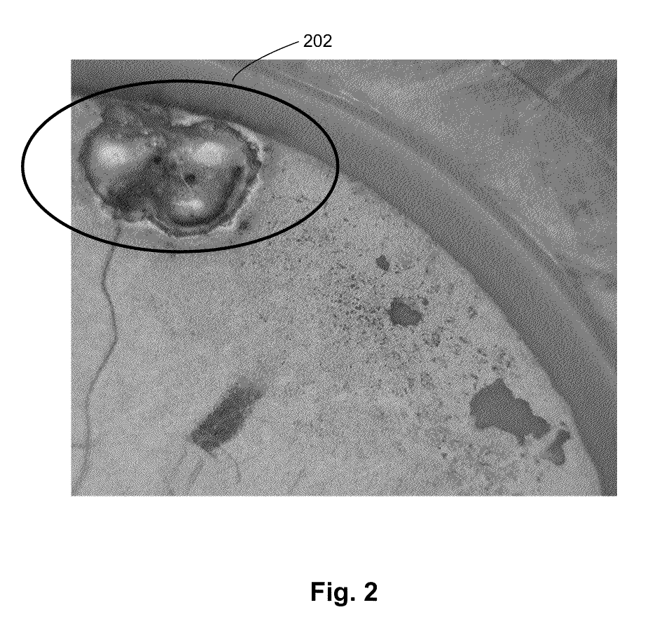

For resonant frequencies greater than 6 MHz, equation 2 requires that the piezoelectric element be less than 350 microns. However, an element this thin is not able to withstand the high voltages required to produce the high-power intensity necessary in some broad-area treatment applications. This is particularly true at power intensities sufficient to achieve hair removal, which may range between 150 and 1000 W/cm². FIG. 2 depicts a catastrophic failure of a piezoelectric element (within indicated region 202) due to a drive voltage beyond the capabilities of the dielectric material. In addition to being susceptible to electrical failure, thin piezoelectric elements are fragile and prone to fracture during manufacturing and assembly.

### 3. Driving a Piezoelectric at Harmonic Frequencies

One solution to problems related to using thin piezoelectric elements is to use a thicker piezoelectric element and drive it using a multiple (harmonic) of its fundamental resonance frequency. In particular, a thicker piezoelectric element allows for higher-drive voltages and is structurally more durable than a thinner element. These are both desirable qualities in a high-power, broad-area ultrasonic device used for hair removal.

In one example, an element with a 3.3 MHz fundamental resonant frequency can be driven at three times (3×) its fundamental resonance to achieve a 10 MHz output. Similarly, an element with a 2 MHz fundamental resonant frequency can be driven at its fifth harmonic (5×) to achieve a 10 MHz output. In general, the thickness of the piezoelectric element may be determined using equation 2 for a given fundamental resonant frequency. Table 2, below, compares maximum-drive voltage for piezoelectric elements of different thicknesses.

TABLE 2

| | | Harmonic | Drive voltage limit |
|---|---|---|---|
| Fundamental | Thickness | resonance | for 0.275 to 0.39 kV/mm |
| frequency | for Nₜ = 2000 | to produce | dielectric strength |
| (MHz) | (μm) | 10 Mhz output | (V) |
| 10 | 200 | 1 | 55 to 78 |
| 3.3 | 600 | 3 | 165 to 234 |
| 2 | 1000 | 5 | 275 to 390 |

In dermatologic applications, operating power intensities may be as low as 150 W/cm² and in some applications exceed 300 W/cm². For a representative piezoelectric transducer with a beam area of 2.4 cm² and a magnitude of electrical impedance of 6.67 Ohms, the drive voltage easily meets or exceeds the damage limit for a fundamental device. In comparison, a harmonic device can operate within the desired parameter range of the representative piezoelectric without a significant risk of piezoelectric damage. Table 3, below, lists the required drive voltage for a representative piezoelectric transducer driven at 10 MHz.

TABLE 3

| | Electrical drive power | |
|---|---|---|
| Acoustic power intensity | at 50% efficiency | Required drive voltage |
| (W/cm²) | (W) | (V) |
| 150 | 720 | 69 |
| 300 | 1440 | 98 |

An additional advantage of using multiple harmonics of a piezoelectric transducer is that a user can select one of a few possible choices of operating frequencies and thereby tailor the device for the desired treatment. In some embodiments, a power supply or other drive circuitry can be configured to selectively deliver the fundamental frequency and/or one or more harmonic frequencies. Higher-frequency ultrasonic beams are more attenuated by a tissue medium than lower-frequency ultrasonic beams and, therefore, penetrate less deeply into the tissue. Based on the depth of penetration, a higher-frequency beam may be desirable in therapeutic applications that are directed to the epidermal and dermal layers of the skin tissue. In general, a high-frequency beam may be appropriate for treating tissue near the application surface of the device. This allows for shallow treatment without propagating large amounts of energy through underlying bone or muscle tissue. Conversely, a lower-frequency beam can be used to treat tissue layers that are relatively thick. Lower-frequency beams may also be appropriate where a more uniform energy distribution through the tissue is desirable.

### 4. Impedance Matching for Multiple Harmonics

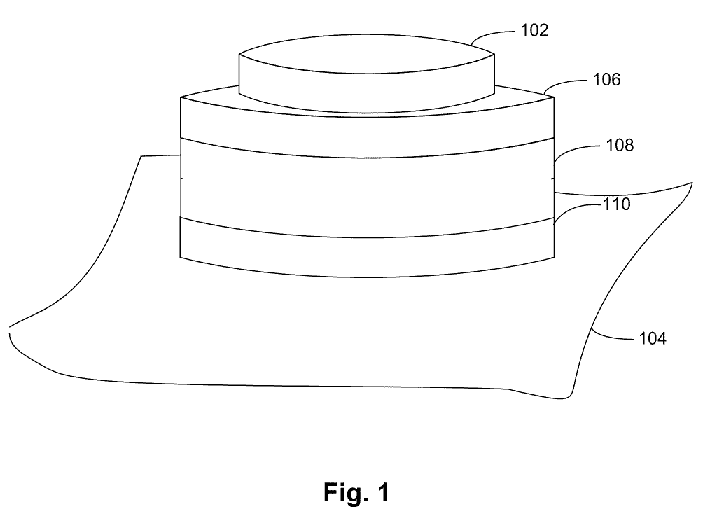

As described above, a piezoelectric transducer transmits an ultrasonic-energy beam into a skin tissue using a transmitter mass having an applicator surface. FIG. 1 depicts an exemplary embodiment of an ultrasonic transducer system 100.

FIG. 1 depicts an ultrasonic transducer system 100 with a single piezoelectric element 102. The piezoelectric element 102 mechanically interfaces with a transmitter mass including one or more impedance-matching layers 106, 108, and 110. One face of impedance-matching layer 108 mechanically interfaces with the surface of a skin tissue 104.

The impedance matching layers 106, 108, and 110 are configured to reduce the back reflection of ultrasound energy similar to an antireflection optical coating. The thickness of these layers is designed to cause destructive interference with back-reflected energy. In one preferred embodiment, a single impedance-matching layer is used and is preferably formed from aluminum. In embodiments using multiple layers (as depicted in FIG. 1), an aluminum impedance-matching layer would be mechanically interfaced with one or more other impedance-matching layers having a different acoustic impedance in much the same way high-index and low-index materials are used in an optical antireflection coating. The other impedance-matching layers may be formed from another material, such as glass, epoxies, polymers including Teflon and PTFE, and metals including copper, brass, and steel. The thicknesses of impedance-matching layers made from different material layers would typically be different from each other since the wavelength in a material depends on the material properties.

In a device using a central impedance-matching layer, the material may be selected so as to provide an impedance (Z) equal to the geometric mean of the impedances of the first (Z₁) and second (Z₂) media interfaced with the impedance matching layers. In general, the impedance (Z) can be calculated as:

Z=\sqrt{Z₁×Z₂}. (Equation 3)

For example, as shown in FIG. 1, the impedance (Z) of a central impedance matching layer 108 may be selected using, for example, Z₁=30 Mrayl for a ceramic piezoelectric element 102 and Z₂=1.6 Mrayl for skin tissue 104. (Note, Z₂=1.5 Mrayl for water.) An ultrasonic device may also use a second impedance matching layer 108 interfacing between the piezoelectric element 102 and the central impedance matching layer 108. The material of impedance matching layer 106 may be selected so as to provide an impedance near the geometric mean of the impedance (Z) of the central matching layer 106 and the impedance of the piezoelectric element 102. Additional impedance matching layers, such as 110, can be designed to have an appropriate impedance in a similar fashion.

The impedance matching layers must also be selected so as to minimize acoustic reflections at the layer interfaces. Quarter-wave matching layers are commonly used in optics and electronic engineering to reduce reflection and thereby improve transmission between materials with different impedances. Designing a matching layer with an appropriate thickness allows reflections from the front and the back face of the impedance matching layer to cancel each other out as they are 180 degrees out of phase. In general, the thickness of an impedance matching layer (tᵢₘₗ) can be a quarter-wavelength of the transmitted frequency (λ):

tᵢₘₗ=λ/4. (Equation 4)

Selecting a thickness (tᵢₘₗ) according to equation 4 allows the layer to function as an impedance matching layer at the transmitted frequency, thereby reducing losses due to internal wave reflection.

Ultrasonic transducers are most efficient when driven at the fundamental resonant frequency of the piezoelectric element or a harmonic thereof. In general, the thickness of an impedance matching layer can be a quarter-wave of any odd harmonic of the fundamental frequency (fᵣₑₛ) and produce enhanced ultrasound output at that harmonic. However, for many solutions, this will result in a transducer device that is substantially optimized at one harmonic and not at others. For example, a quarter-wavelength of the third harmonic (¼·λ_{3rd}) is not the same as a quarter-wavelength of the fifth harmonic (¼·λ₅ₜₕ). However, it is possible to design an impedance matching layer that features a reduced internal reflection for more than one odd harmonic. Indeed, for any fundamental resonance, there is a subset of solutions for the impedance matching layer thickness that will be resonant at multiple odd harmonics. This is a critical feature for efficient transducer operation at multiple harmonic frequencies.

First, the fundamental frequency (fᵣₑₛ) is used to compute the wavelength (λᵢ) of the ultrasonic wave that is transmitted through the impedance matching layer (i). For each impedance matching layer (i),

λᵢ=cᵢ/fᵣₑₛ, (Equation 5)

where cᵢ is the speed of sound in the material used to form the impedance matching layer (i). The thickness (tᵢ) of the impedance matching layer (i) can be calculated as:

tᵢ=(2n−1)·λᵢ/4, (Equation 6)

for a positive integer (n). Equation 6 can be used to calculate the thickness for any number of impedance matching layers used in a transducer device. In practice, the actual thickness of an impedance matching layer may deviate from t, by up to 10% provided some loss in efficiency can be tolerated.

### 5. Exemplary Embodiment

A preferred embodiment of an ultrasonic transducer device is capable of providing a relatively high-power, short-duration energy burst in an efficient and reliable manner. In particular, the device should be capable of providing an ultrasonic beam with a power intensity at least 150 W/cm² to as much as 1000 W/cm² over a duration of more than 5 milliseconds and less than 100 milliseconds. In a preferred embodiment, the beam intensity is greater than or equal to 300 W/cm² and is applied over a duration that is greater than 5 milliseconds and less than 75 milliseconds. In a more preferable embodiment, the beam is applied over a duration greater than 5 milliseconds and less than 50 milliseconds.

To facilitate treatment of body hair, the device distributes the power intensity over a broad-area beam to irradiate a larger portion of a skin surface. In preferred embodiments, the beam area is greater than or equal to 4 mm². In a more preferable embodiment, the beam area is greater than or equal to 16 mm². In the most preferable embodiment, the beam area is greater than or equal to 50 mm².

Irradiating tissue using a high-power ultrasonic output over a broad area requires a relatively high drive voltage for the piezoelectric element. For example, in some applications, a drive voltage greater than 55 volts is required. In some applications, the drive voltage may be greater than 70 volts. The maximum voltage will determine the minimum thickness of the piezoelectric element based on the dielectric strength of the piezoelectric material. Depending on the material used, the dielectric strength of the piezoelectric material may be somewhere between 0.275-0.390 kV/mm. Accordingly, the minimum thickness of the piezoelectric element will be approximately 250 microns for an exemplary material with a dielectric strength of 0.275 kV/mm. The thickness, in turn, determines the fundamental resonant frequency of the piezoelectric element as shown in equation 1. In a preferred embodiment, the fundamental frequency is 5 MHz or less. In a more preferred embodiment, the fundamental frequency is between 1 and 4 MHz.

To achieve the high frequency irradiation desirable in treatments such as hair removal, the piezoelectric element may be operated at a harmonic of the fundamental resonant frequency. In preferred embodiments, a device uses frequencies between 5 and 20 MHz. In a more preferred embodiment, a device uses frequencies between 7 and 15 MHz.

The embodiment depicted in FIG. 1 includes a single piezoelectric element 102 mechanically interfaced with a transmitter mass made from one or more impedance matching layers 106, 108, and 110. The piezoelectric element 102 and the impedance matching layers (106, 108, and 110) may be mechanically bonded using a compatible adhesive. Preferably, the impedance matching layers are designed to provide efficient power transmission at several frequencies. For example, the high efficiency may be achieved by designing matching layers to be resonant at a quarter-wavelength multiple of each of the desired output harmonics as shown in equation 6. In general, it is desirable to keep the integer (n) as low as possible to reduce losses due to attenuation in the impedance matching layer material.

### 6. Empirical Method for Determining Impedance Matching Layer Thickness

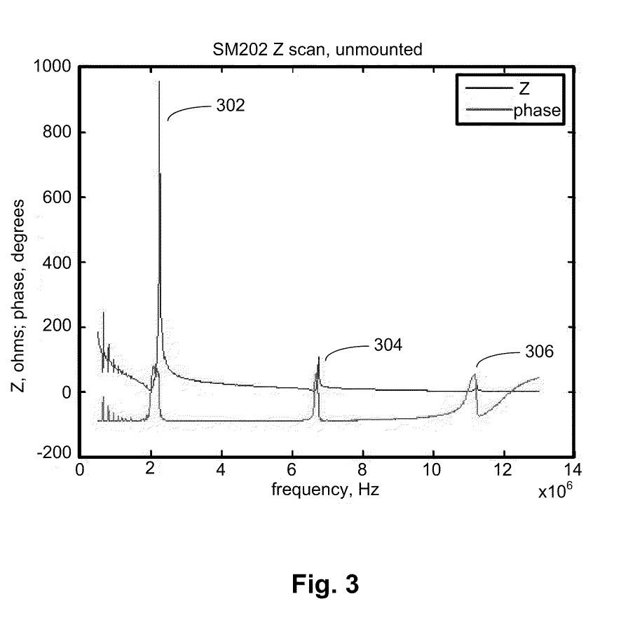

The thickness of the impedance matching layers can also be determined using an empirical method. FIG. 3 depicts the electrical impedance of a piezoelectric element measured over a range large enough to encompass at least the third harmonic of the fundamental frequency. Each peak in the impedance plot (302, 304, and 306) represents either the fundamental or an odd harmonic resonance of the piezoelectric element.

The frequency corresponding to each peak in the impedance plot is used to calculate a corresponding wavelength (λₕₐᵣₘ) based on the speed of sound in the material. A series of impedance matching thicknesses are calculated according to:

t=(2n−1)·λₕₐᵣₘ/4, (Equation 7)

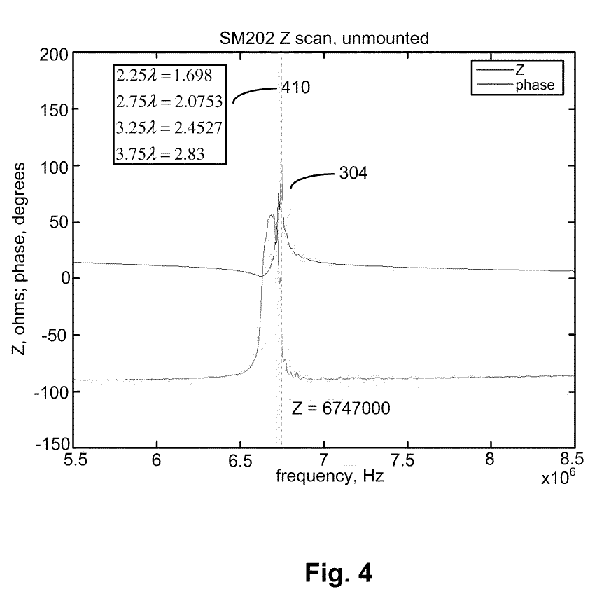

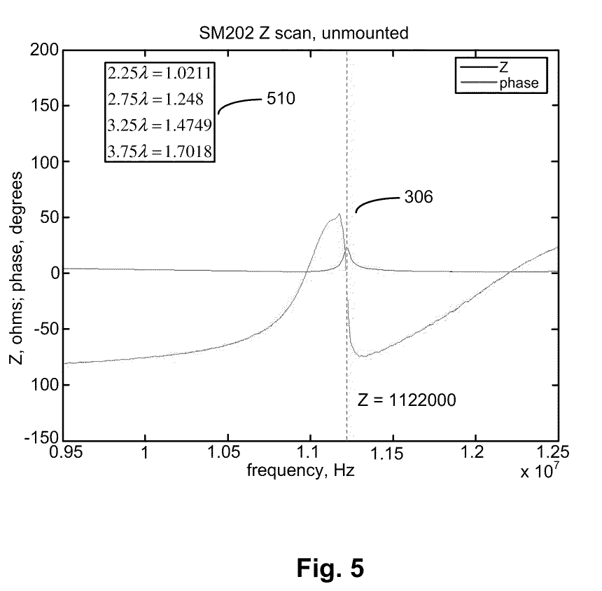

FIG. 4 depicts the peak impedance 304 at the third harmonic frequency 6.747 MHz corresponding to a third harmonic wavelength of 2.251 MHz. A series of thicknesses are calculated as shown in the table 410. Similarly, FIG. 5 depicts the peak impedance 306 at the fifth harmonic frequency 11.220 MHz corresponding to a fifth harmonic wavelength of 2.251 MHz. A series of thicknesses are calculated as shown in the table 510.

The series of thicknesses calculated for each of the higher harmonic peaks are compared, and common thicknesses are selected. Due to manufacturing tolerances and measurement error, the common thicknesses may not be exactly the same value. In this example, the common result is approximately 1.7 mm corresponding to n=9 for the third harmonic (FIG. 4) and n=15 for the fifth harmonic (FIG. 5). Note, 1.7 mm also corresponds to a ¾ wavelength thickness of the fundamental resonant frequency 2.25 MHz shown as the first impedance peak 302 in FIG. 3.

### 6. Exemplary Ultrasonic Transducer System

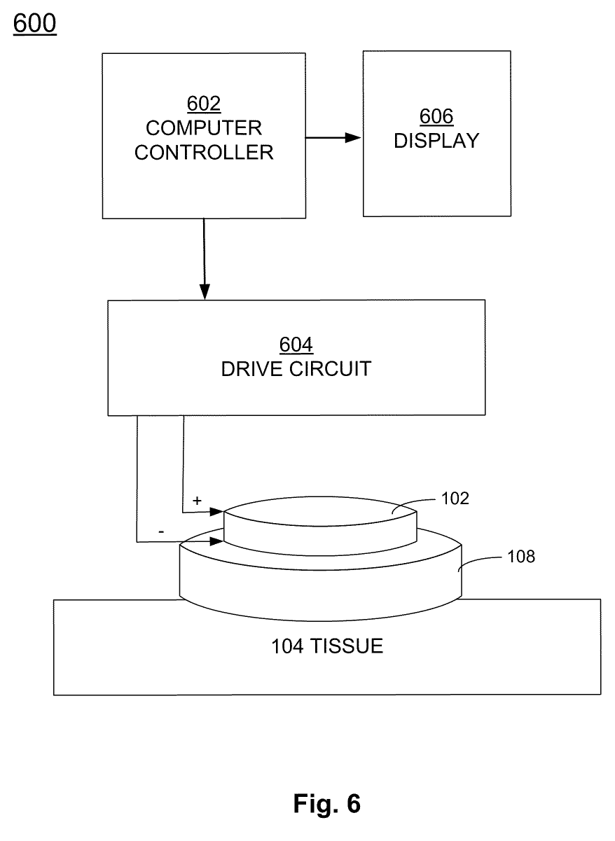

FIG. 6 depicts an exemplary system 600 using an ultrasonic transducer device for hair removal in a skin tissue 104. In one embodiment, one or more piezoelectric elements 102 are used to irradiate the skin tissue 104 with an ultrasonic energy beam. The beam is transmitted to the skin tissue 104 via at least one impedance matching layer 108. The piezoelectric element 102 and impedance matching layer 108 may be mounted in the housing of a hand-held dermatological treatment device (not shown).

A drive circuit 604 is used to produce the excitation voltage for the one or more piezoelectric elements 102. As shown in FIG. 6, the drive circuit may drive the piezoelectric element 102 using a pair of electrodes. The drive circuit 604 may be a waveform generation device suitable for delivering an ultrasonic frequency voltage. In some embodiments, more than one waveform-generation device is used as the drive circuit 604. In some embodiments, the drive circuit 604 may be controlled by a computer controller 602. In some embodiments, the drive circuit 604 includes an internal controller in addition to, or instead of, computer controller 602. In a preferred embodiment, it is possible to set the drive circuit 604 to more than one excitation frequency and more than one treatment duration time.

The computer controller 602 may include one or more processors for executing computer-readable instructions. The computer-readable instructions allow the computer to control the drive circuit 604 to produce one or more drive frequencies at one or more drive voltages. The computer controller may also include computer memory, such as read-only memory (ROM), random-access memory (RAM), and one or more non-volatile storage media drives for storing computer-readable instructions or programs. The computer controller may be equipped with a computer display 606 or other visual read-out device.

It should be appreciated that the various features of the embodiments that have been described may be combined in various ways to produce numerous additional embodiments. Accordingly, the invention is not to be limited by those specific embodiments and methods described herein.

I claim:

1. A hand-held dermatological treatment device comprising:
an ultrasound transducer having a fundamental resonant frequency,
at least one matching layer mechanically interfaced to an output surface of the ultrasonic transducer, said at least one matching layer having a thickness about equal to an odd multiple of a quarter wavelength of the fundamental frequency; and
a drive circuit for applying an electrical power input to the ultrasonic transducer, the electrical power input having:
a drive frequency that is an odd harmonic of the fundamental resonant frequency of the ultrasonic transducer, and
a drive voltage greater than 55 volts,
wherein the electrical power input results in an ultrasonic transducer output with a power intensity greater than 150 W/cm².

2. The dermatological treatment device of claim 1, wherein the drive circuit is configured to selectively apply at least two odd harmonic frequencies of the fundamental resonant frequency of the ultrasonic transducer.

3. The dermatological treatment device of claim 1, wherein the electrical power input is applied for less than 100 milliseconds.

4. The dermatological treatment device of claim 1, wherein the electrical power input is applied for less than 50 milliseconds.

5. The dermatological treatment device of claim 1, wherein the odd harmonic of the fundamental frequency is between 5 and 20 MHz.

6. The dermatological treatment device of claim 1, wherein the fundamental frequency is 5 MHz or less and the odd harmonic of the fundamental frequency is 5 MHz or higher.

7. The dermatological treatment device of claim 1, wherein the odd harmonic frequency is between 5 and 20 MHz and the electrical power input is applied for less than 100 milliseconds.

8. A method of removing hair from a person's skin with ultrasound energy comprising the steps of:
positioning a dermatological treatment device against the skin, said device including an ultrasound transducer, said ultrasonic transducer having a fundamental resonant frequency; and
driving the ultrasonic transducer with an electrical input having a frequency which is an odd harmonic frequency of the fundamental frequency.

9. The method of claim 8, wherein the electrical input has a drive voltage greater than 55 volts and results in an ultrasonic energy output with a power intensity greater than 150 W/cm².

10. The method of claim 8, wherein driving the ultrasonic transducer further comprises selectively applying at least two odd harmonic frequencies of the fundamental resonant frequency of the transducer.

11. The method of claim 8, wherein the electrical power input is applied for less than 100 milliseconds.

12. The method of claim 8, wherein the odd harmonic of the fundamental frequency is between 5 and 20 MHz.

13. The method of claim 8, wherein the fundamental frequency is 5 MHz or less and the odd harmonic of the fundamental frequency is 5 MHz or higher.

14. The method of claim 8, wherein the odd harmonic frequency is between 5 and 20 MHz and the electrical power input is applied for less than 100 milliseconds.

15. A method of removing hair from a person's skin with ultrasound energy comprising the steps of:
positioning a dermatological treatment device against the skin, said device including an ultrasound transducer, said ultrasonic transducer having a fundamental resonant frequency; and
driving the ultrasonic transducer with an electrical input having:
a frequency which is an odd harmonic frequency of the fundamental frequency, and
a drive voltage greater than 55 volts, wherein the electrical power results in an ultrasonic transducer output with a power intensity greater than 150 W/cm².

16. The method of claim 15, wherein driving the ultrasonic transducer further comprises selectively applying at least two odd harmonic frequencies of the fundamental resonant frequency of the transducer.

17. The method of claim 15, wherein the electrical power input is applied for less than 100 milliseconds.

18. The method of claim 15, wherein the odd harmonic of the fundamental frequency is between 5 and 20 MHz.

19. The method of claim 15, wherein the fundamental frequency is 5 MHz or less and the odd harmonic of the fundamental frequency is 5 MHz or higher.

20. The method of claim 15, wherein the odd harmonic frequency is between 5 and 20 MHz and the electrical power input is applied for less than 100 milliseconds.
